(19) United States
(12) Patent Application Publication
(10) Pub. No.: US 2013/0005780 A1
(43) Pub. Date: Jan. 3, 2013

(54) CONTROLLED RELEASE PHARMACEUTICAL COMPOSITIONS OF TAPENTADOL

(75) Inventors: Kumodini Kashinath Mahakal, Pune (IN); Pal Singh, Pune (IN); Purna Chandra Ray, Pune (IN)

(73) Assignee: LUPIN LIMITED, Mumbai (IN)

(21) Appl. No.: 13/581,981

(22) PCT Filed: Feb. 16, 2011
(86) PCT No.: PCT/IB11/00287

§ 371 Date: Aug. 30, 2012

(30) Foreign Application Priority Data
Mar. 1, 2010 (IN) 195/KOL/2010

(51) Int. Cl. C07D 213/53; C07C 227/40; A61K 31/44

(52) U.S. Cl. 514/357; 546/332; 562/567

(57) ABSTRACT

The present invention provides atazanavir sulfate substantially free of diastereomeric impurities. The present invention also provides atazanavir sulfate having D-tertiary leucine analogues less than 0.1%. The present invention further relates to an improved process for preparing atazanavir sulfate, substantially free of its diastereoisomeric impurities, which comprises of reacting diamino compound (IV) with N-methoxycarbonyl-(L)-tertiary-leucine (V) having D-isomer less than 0.1% to obtain atazanavir base; conversion of atazanavir base to atazanavir sulfate by reacting with sulfuric acid and crystallization of atazanavir sulfate from suitable organic solvent(s).

### FIELD OF THE INVENTION

The present invention is related to atazanavir sulfate substantially free of its diastereomeric impurities and process for its preparation.

### BACKGROUND OF THE INVENTION

The human immunodeficiency virus (HIV) is responsible for the pathogenesis of the acquired immunodeficiency disease syndrome (AIDS) in human beings. It has been found that a functional viral protease (HIV protease), which is an enzyme responsible for the processing of poly-proteins to structural proteins and viral enzymes, is essential for the maturation of viral particles to a fully infectious virus. Therefore, HIV protease has become a target of choice for an effective AIDS therapy. Clinical studies with HIV protease inhibitors, as single therapy or in combination with reverse transcriptase inhibitors, has shown excellent efficacy in AIDS patients.

Atazanavir is an acyclic aza-peptidomimetic and one of the potent HIV protease inhibitor. Its sulfate salt has better bioavailability than the free base, with a half-life suitable for once-daily dosing.

Atazanavir sulfate is marketed under the name of REYATAZ and is indicated in combination with other antiretroviral agents for the treatment of HIV-1 infection. REYATAZ capsules are available for oral administration in strengths containing the equivalent of 100 mg, 150 mg, 200 mg, or 300 mg of atazanavir as atazanavir sulfate.

Atazanavir sulfate is chemically known as (3S,8S,9S,12S)-3,12-bis(1,1-dimethylethyl)-8-hydroxy-4,11-dioxo-9-(phenylmethyl)-6-[[4-(2-pyridinyl)phenyl]-2,5,6,10,13-pentaazatetradecanedioic acid dimethyl ester, sulfate (1:1), and it is represented by the following structure:

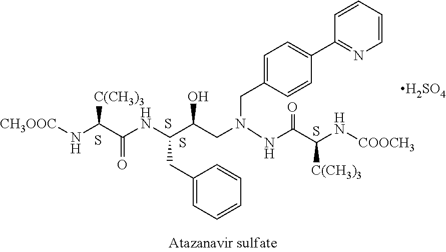

From the chemical structure it is evident that it has four chiral centres which result in total of 16 stereo-isomers. Atazanavir is SSSS isomer; it has S configuration in all of its four chiral centres.

The process for preparation of atazanavir base as shown in Scheme-I is described in U.S. Pat. No. 5,849,911, U.S. Pat. No. 6,300,519, Guido Bold et al., Journal of Medicinal Chemistry, 1998, Vol. 41, No. 18, 3387-3401 and Drugs of the Future, 1999, 24 (4), 375-380, wherein the amino protecting group is tert-butoxycarbonyl and the condensation of diamino compound (IV) with N-methoxycarbonyl-(L)-tertiary-leucine (V) is achieved by using O-(1,2-dihydro-2-oxo-1-pyridyl)-N,N,N′,N′-tetramethyluronium tetrafluoroborate (TPTU) in dichloromethane or dimethylformamide.

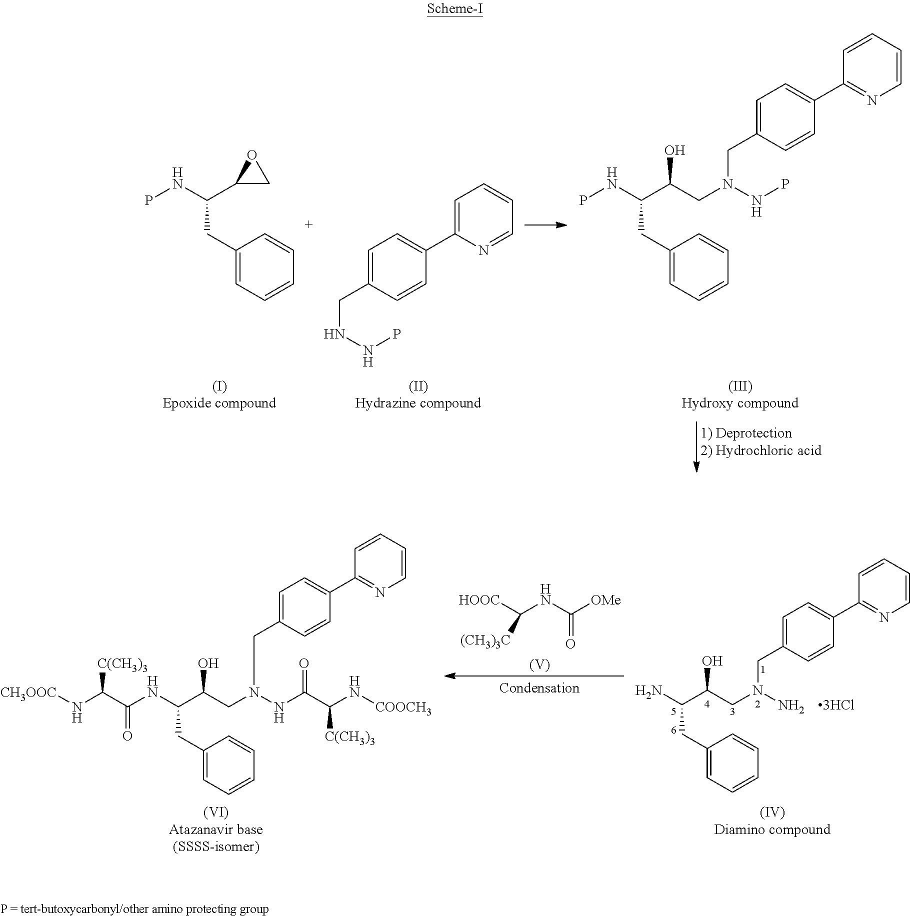

Zhongmin Xu et al., Organic Process Research & Development, 2002, 6, 323-328, describe similar conversion of diamino compound (IV) to atazanavir base (VI) by reacting with N-methoxycarbonyl-(L)-tertiary-leucine (V) using water soluble carbodiimide, 1-hydroxy-benzotriazole in dichloromethane as shown in Scheme-I.

The PCT application WO 2008065490 A2 describes a process for the preparation of atazanavir as in scheme-II, which comprises of reacting the hydrochloride salt of amino compound (VII) with N-methoxycarbonyl-L-tert-leucine (V) in the presence of dicyclohexylcarbodiimide (DCC), 1-hydroxy-benzotriazole (HOBT) followed by the removal of benzyloxycarbonyl group and then the reaction of subsequent intermediate (IX) with methyl chloroformate.

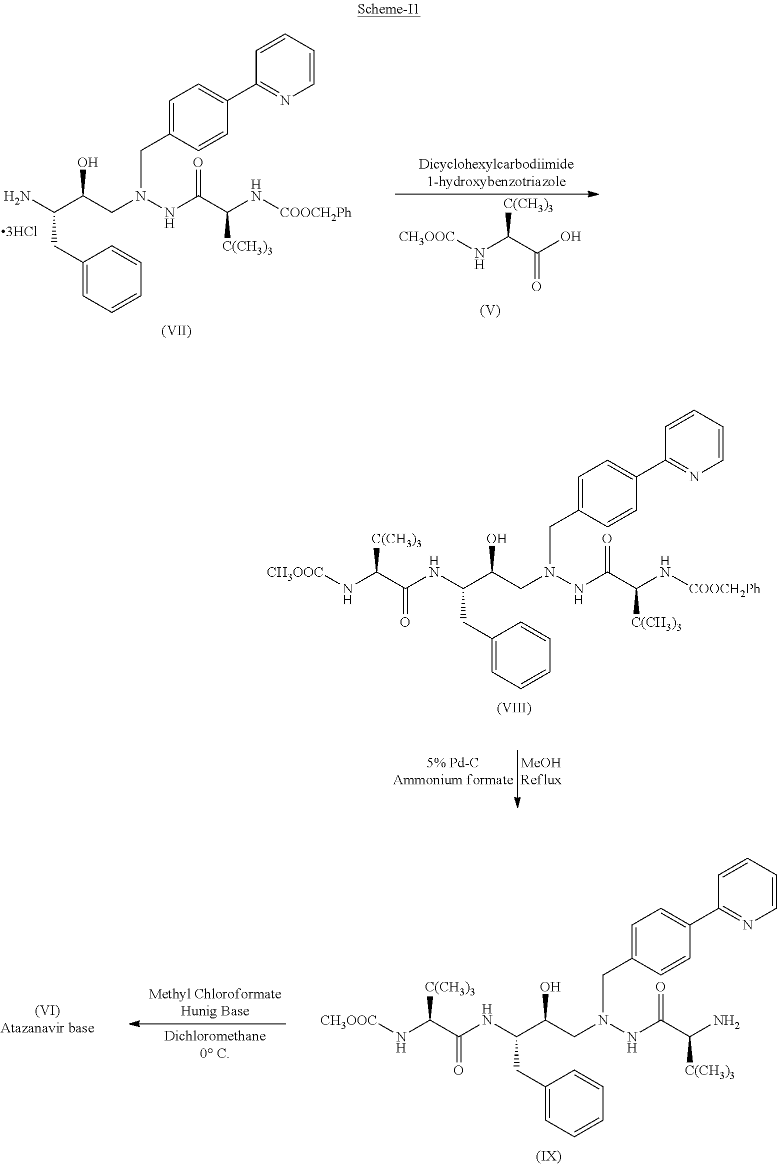

Xing Fan et al., Organic Process Research & Development, 2008, 12, 69-75, discloses alternate synthesis employing the diastereoselective reduction of ketomethylene aza-dipeptide (XII) as the final step. The coupling of the two intermediates, N-(methoxycarbonyl)-L-tert-leucine acylated benzyl hydrazine (X) and chloromethyl ketone (XI) furnished the amino ketone (XII) as shown in scheme-III.

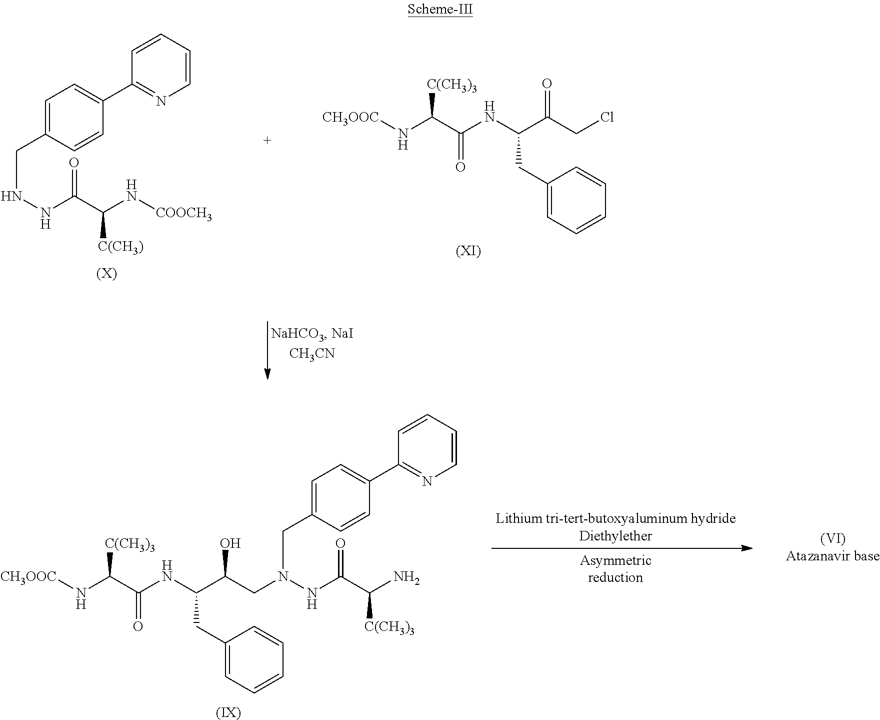

The U.S. Pat. No. 6,110,946 covers various intermediates used in schemes I to IV;

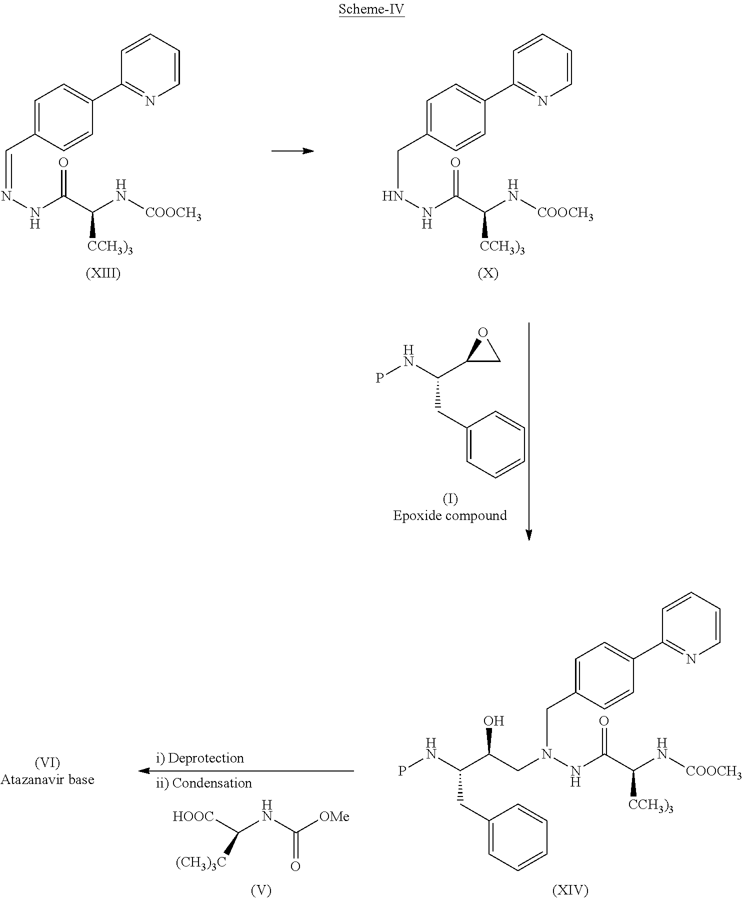

The example 3 of the U.S. Pat. No. 6,087,383 to Singh et al. describes the preparation of atazanavir sulfate by reacting atazanavir base with dilute sulfuric acid in suitable solvent. It further describes two crystalline forms of atazanavir sulfate, one as Type-II crystal which is hydrated hygroscopic and another as Type-I crystal which appear to be an anhydrous/desolvated crystalline form.

It is always desirable to prepare pharmaceutical products of a high purity having a minimum amount of impurities, in order to reduce adverse side effects and to improve the shelf life of active ingredient, as well as its formulation. In some cases it has been found that high purity also facilitates in formulation process.

Therefore, the present invention is directed to provide an improved synthetic process for the preparation of atazanavir, having minimum amount of impurities.

### OBJECTS AND SUMMARY OF THE INVENTION

The objective of the present invention is to provide atazanavir sulfate that is substantially free of diastereomeric impurities.

Another objective of the present invention relates to an improved process for preparing atazanavir sulfate, substantially free of its diastereoisomeric impurities, which comprises of reacting diamino compound (IV) with N-methoxycarbonyl-(L)-tertiary-leucine (V) having D-isomer less than 0.1% to obtain atazanavir base; conversion of atazanavir base to atazanavir sulfate by reacting with sulfuric acid and crystallization of atazanavir sulfate from suitable organic solvent(s).

The process for preparing atazanavir sulfate of the present invention is as shown in the Scheme-I. The reaction of hydrazine compound (II) with the epoxide compound (I) gives hydroxy compound (III) which on deprotection and treatment with aqueous hydrochloric acid gives hydrochloride salt of diamino compound (IV).

N-(methoxycarbonyl)-L-tert-leucine (V) is prepared by reaction of L-tertiary-leucine, having D-isomer less than 0.5% with methyl chloroformate and then subjected to purification by crystallization from ethyl acetate-n-heptane mixture. N-(methoxycarbonyl)-L-tert-leucine (V) having D-isomer less than 0.1% is used for preparation of atazanavir sulfate.

The diamino compound (IV) is reacted with N-(methoxycarbonyl)-L-tert-leucine (V) in the presence of 1-hydroxy-benzotriazole (HOBT) and water soluble carbodiimide, such as 1-(3-dimethylaminopropyl)-3-ethylcarbodiimide hydrochloride (EDC) in the presence of organic tertiary-amine to obtain atazanavir base (VI). To the solution of atazanavir base (VI) in alcohol, concentrated sulfuric acid is added, followed by n-heptane, to obtain atazanavir sulfate.

The process of the present invention affords the atazanavir sulfate that has diastereomers less than 0.2%.

### DETAILED DESCRIPTION

The present invention provides atazanavir sulfate substantially free of diastereomeric impurities. The present invention provides atazanavir sulfate with D-isomeric impurities (RSSS isomer, SSSR isomer and RSSR isomer) less than 0.2%, preferably less than 0.1%, most preferably less than 0.05%, measured as area percentage by HPLC.

The present invention further relates to an improved process for preparing atazanavir sulfate which is substantially free of its diastereoisomeric impurities. The process of the present invention affords the atazanavir sulfate that has diastereomers less than 0.2%, preferably less than 0.1%, most preferably less than 0.05%, measured as area percentage by HPLC.

The process for preparing atazanavir sulfate of the present invention is as shown in Scheme-I.

The reaction of epoxide compound (I) with hydrazine compound (II) in lower alcohols gives the hydroxy compound (III). Lower alcohol used information of hydroxy compound (III) include methanol, ethanol, isopropanol and n-butanol, preferably isopropanol.

The hydroxy compound (III) was subjected to amino group deprotection followed by treatment with concentrated hydrochloric acid to give hydrochloride salt of diamino compound (IV).

N-(methoxycarbonyl)-L-tert-leucine (V) is prepared by reaction of L-tertiary-leucine, having D-isomer less than 0.5% with methyl chloroformate and then subjected to purification by crystallization from ethyl acetate-n-heptane mixture. N-(methoxycarbonyl)-L-tert-leucine (V) having corresponding D-isomer less than 0.1% is selected and used for preparation of atazanavir sulfate. The crystallization of N-(methoxycarbonyl)-L-tert-leucine (V) is repeated till D-isomer is less than 0.1%.

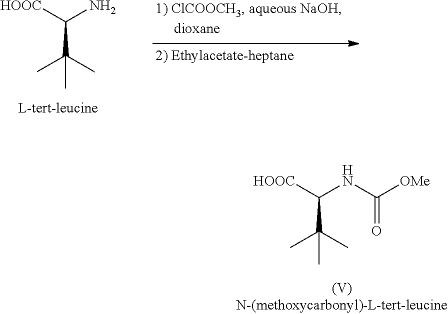

The diamino compound (IV) is condensed with N-(methoxycarbonyl)-L-tert-leucine (V) of chiral purity more than 99.9% in the presence of 1-hydroxy-benzotriazole (HOBT) and water soluble carbodiimide, such as 1-(3-dimethylaminopropyl)-3-ethylcarbodiimide hydrochloride (EDC) in the presence of organic tertiary-amine to obtain atazanavir base (VI).

Each amino group in diamino compound (IV) reacts with one molecule of N-methoxycarbonyl-(L)-tertiary-leucine (V) to afford SSSS isomer which is the required compound.

The condensation of diamino compound (IV) with N-methoxycarbonyl-(L)-tertiary-leucine (V) is carried out in the presence of 1-hydroxy-benzotriazole (HOBT), water soluble carbodiimide and organic tertiary amine in biphasic mixture of water immiscible solvent and water. The solution of N-methoxycarbonyl-(L)-tertiary-leucine (V) is made in suitable water immiscible solvent such as halogenated hydrocarbons like dichloromethane (DCM), chloroform, dichloroethane; esters like ethyl acetate, propyl acetate, butyl acetate; aromatic solvents like benzene, toluene, xylene, ethylbenzene, chlorobenzene; ethers like diethyl ether, diisopropyl ether (DIPE), methyl tert-butylether (MTBE), tetrahydrofuran (THF), dioxane; preferred solvent is dichloromethane.

The carbodiimides that are used in the condensation of diamino compound (IV) with N-methoxycarbonyl-L-tertiary-leucine (V) can be selected from dicyclohexyl carbodiimide (DCC), diisopropyl carbodiimide (DIC), 1-(3-dimethylaminopropyl)-3-ethyl carbodiimide (EDC) and carried out in the presence of 1-hydroxy-benzotriazole (HOBT) and in the presence of organic tertiary-amine in suitable solvent. Most preferably the water soluble carbodiimide such as 1-(3-dimethylaminopropyl)-3-ethyl carbodiimide (EDC) is used.

Suitable solvent for condensation are selected from halogenated hydrocarbons like dichloromethane (DCM), chloroform, dichloroethane; amides like dimethyl acetamide (DMA), dimethyl formamide (DMF); esters like ethyl acetate, propyl acetate, butyl acetate; ethers like diethyl ether, diisopropyl ether (DIPE), methyl tert-butylether (MTBE), tetrahydrofuran (THF), dioxane; aromatic solvents like benzene, toluene, xylene, ethylbenzene, chlorobenzene; ketones like acetone, methyl isobutyl ketone (MIBK), methylethyl ketone (MEK); nitriles like acetonitrile and propionitrile; and mixtures thereof.

The other carbonyl activating reagents such as 1-hydroxy-aza-benzotriazole (HOAT), 4-(N,N-dimethylamino)pyridine (DMAP) can also be used for condensation.

The condensation can be also carried out with phase transfer catalysts such as tetramethylammonium bromide, phenyltrimethylammonium bromide, tetra-n-butylammonium bromide, (1-butyl)triethylammonium bromide and the like.

The atazanavir sulfate can be prepared in solvents selected from acetonitrile, acetone, ethanol and heptane or mixtures thereof. Preferably, to the solution of atazanavir base (VI) in ethanol, concentrated sulfuric acid is added followed by n-heptane to obtain atazanavir sulfate.

The innovators of the present invention have found that contamination of D-isomer in N-methoxycarbonyl-(L)-tertiary-leucine (V) leads to formation of various diastereomeric impurities.

The impurity of D-tertiary leucine in the (L)-tertiary-leucine converts to corresponding N-methoxycarbonyl-(D)-tertiary-leucine, which in turn leads to D-tertiary leucine analogous impurities in Atazanavir.

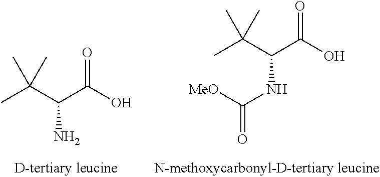

When one of the two amino groups of diamino compound (IV) reacts with N-methoxycarbonyl-(D)-tertiary-leucine, and other amino group of diamino compound (IV) reacts with N-methoxycarbonyl-(L)-tertiary-leucine (V) then it leads to formation of RSSS impurity or SSSR impurity.

When the 2-amino group of diamino compound (IV) reacts with N-methoxycarbonyl-(D)-tertiary-leucine, it leads to formation of RSSS isomer.

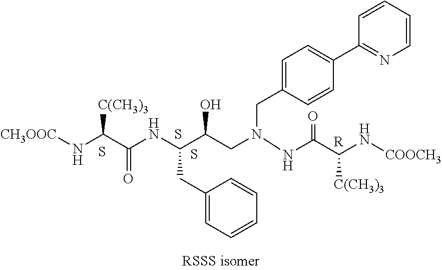

Chemical name: (3R,8S,9S,12S)-3,12-Bis(1,1-dimethylethyl)-8-hydroxy-4,11-dioxo-9-(phenylmethyl)-6-[[4-(2-pyridinyl)phenyl]-2,5,6,10,13-pentaazatetradecanedioic acid dimethyl ester

When the 5-amino group of diamino compound (IV) reacts with N-methoxycarbonyl-(D)-tertiary-leucine, it leads to formation of SSSR isomer.

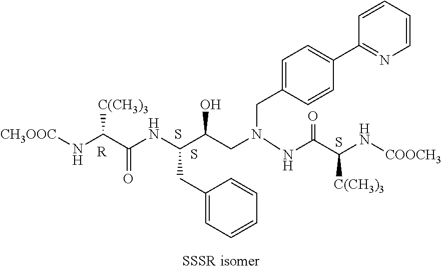

Chemical name: (3S,8S,9S,12R)-3,12-Bis(1,1-dimethylethyl)-8-hydroxy-4,11-dioxo-9-(phenylmethyl)-6-[[4-(2-pyridinyl)phenyl]-2,5,6,10,13-pentaazatetradecanedioic acid dimethyl ester

Moreover, there is possibility of formation of RSSR isomer as an impurity, when both the amino group of diamino compound (IV) react with two different molecules of N-methoxycarbonyl-(D)-tertiary-leucine.

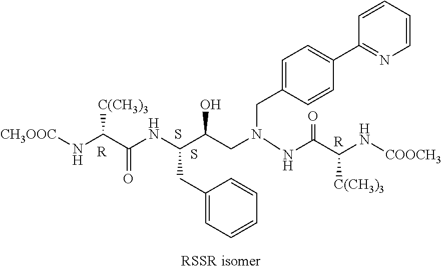

Chemical name: (3R,8S,9S,12R)-3,12-Bis(1,1-dimethylethyl)-8-hydroxy-4,11-dioxo-9-(phenylmethyl)-6-[[4-(2-pyridinyl)phenyl]-2,5,6,10,13-pentaazatetradecanedioic acid dimethyl ester

The publication, Zhongmin Xu et al., Organic Process Research & Development, 2002, 6, 323-328, describe preparation of atazanavir bisulfate with HPLC area purity 99.8%. But this publication does not mention about how to control the diastereomeric impurities in atazanavir base/atazanavir bisulfate.

In one embodiment, the present invention provides atazanavir sulfate substantially free of its diastereomeric isomers. The present invention provides atazanavir sulfate having purity greater than 99.8%, preferably greater than 99.9% by HPLC, most preferably greater than 99.95%, measured as area percentage by HPLC.

In another embodiment, the present invention provides a process for preparation of atazanavir sulfate substantially free of diastereomers comprising the steps:
a) reaction of diamino compound (IV) with N-methoxycarbonyl-L-tertiary leucine (V) having D-isomer less than 0.1% to obtain atazanavir base (VI);
b) optionally purification of atazanavir base (VI); and
c) conversion of atazanavir base (VI) to atazanavir sulfate.

In another aspect, the present invention provides a process wherein the level of D-isomer in N-methoxycarbonyl-L-tertiary-leucine (V), is controlled by selecting a sample of L-tertiary-leucine containing D-isomer less than 0.5% and purifying the N-methoxycarbonyl-L-tertiary-leucine (V) by crystallization.

The process to obtain N-methoxycarbonyl-L-tertiary-leucine with D-isomer less than 0.1% comprises of:
a) selection of L-tertiary-leucine containing D-isomer less than 0.5%;
b) conversion of L-tertiary-leucine to N-methoxycarbonyl-L-tertiary leucine (V); and
c) purification of N-methoxycarbonyl-L-tertiary leucine (V).

N-methoxycarbonyl-L-tertiary leucine (V) can be prepared by treatment of L-tertiary-leucine with methylchloroformate, dimethyldicarbonate and N-methoxycarbonyl-phthalimide etc., preferably methylchloroformate.

The purification of N-methoxycarbonyl-(L)-tertiary-leucine (V) can be achieved by crystallization from solvents selected from halogenated hydrocarbons like dichloromethane (DCM), chloroform, dichloroethane; amides like dimethyl acetamide (DMA), dimethyl formamide (DMF); esters like ethyl acetate, propyl acetate, butyl acetate; ethers like diethyl ether, diisopropyl ether (DIPE), methyl tert-butylether (MTBE), tetrahydrofuran (THF), dioxane; aromatic solvents like benzene, toluene, xylene, ethylbenzene, chlorobenzene; ketones like acetone, methyl isobutyl ketone (MIBK), methylethyl ketone (MEK); nitriles like acetonitrile and propionitrile; and mixtures thereof. Preferably, purification of N-methoxycarbonyl-L-tertiary leucine (V) is carried out by crystallization from ethyl acetate-heptane mixture.

The purification of N-methoxycarbonyl-(L)-tertiary-leucine can also be achieved by other methods such as column chromatography.

The amino protecting group in epoxy compound (I) and hydrazine compound (II) can be selected from tertiary-butoxycarbonyl (BOC), trifluoroacetyl, triphenylmethyl, benzyloxycarbonyl, acetyl, benzyl, benzoyl, p-toluenesulfonyl, trialkyl silyl such as trimethyl silyl and the likes.

The amino group deprotection of the hydroxy compound (III) can be achieved by treatment with suitable reagents at appropriate conditions depending on the amino protecting group used. The reagents used for deprotection of amino group include, but not limited to are trifluoro acetic acid, hydrofluoric acid, hydrochloric acid, acetic anhydride/pyridine, potassium carbonate and hydrogenation in the presence of transition metal catalysts such as Nickel, Palladium, Platinum and Rhodium on charcoal.

The organic tertiary amines referred herein above includes, triethylamine (TEA), tert-butylamine, N,N-diisopropylethyl amine (DIPEA) and the likes; the preferred organic tertiary amine is DIPEA.

The reaction temperature for different steps of the process is in the range −10 to 100° C., preferably 20 to 80° C.

The atazanavir base is optionally purified by crystallization from ethanol-water mixture or by the methods known in the literature.

The tapped density of atazanavir sulfate prepared by process of present invention varies from 0.24-0.29 g/mL and the particle size is d (0.9)-6.5 μm and d (0.5)-1.7 μm.

The presence of impurities in atazanavir sulfate may pose a problem for formulation in that impurities often affect the safety and shelf life of a formulation. Therefore, the atazanavir sulfate prepared by the process of the present invention might be ideal for pharmaceutical formulation, since it is substantially free of the D-tertiary-leucine analogues and other diastereomeric impurities.

It will be apparent to those skilled in the art that various modifications and variations can be made in the present invention and specific examples provided herein, without deviating from the scope of the invention. Therefore, it is intended that the scope of the present invention covers the modifications and/or variations that are equivalents.

### SPECIFIC EXAMPLES

The present invention can be illustrated in one of its embodiments by the following non-limiting examples. The purity of atazanavir sulfate was measured as area percentage by HPLC method having following parameters:
Column: Ascentis Express C18 (4.6×150 mm), 2.7 μm
Mobile Phase: Water: Acetonitrile (55: 45)
Flow rate: 1.0 mL/min
Run time 15 min.
Detection: UV at 250 nm

### Example 1

### Preparation of N-methoxycarbonyl-(L)-tert-leucine from L-tert-leucine (V)

L-tert-leucine (100 g, 0.76 mole, D-isomer=0.35%) was added to a mixture of sodium hydroxide (100 g) in 1250 mL water and methyl chloroformate (144.3 g, 1.5 mole). Heated at 60° C. for 18 hours. Cooled to 25° C., acidified with concentrated HCl and extracted with ethyl acetate. Organic layer was concentrated under reduced pressure to obtain viscous oil. Pure N-methoxy carbonyl-(L)-tert-leucine (0.2 g) and n-heptane added, stirred for 1 hour and solid was filtered.

The wet solid (˜130 g) was stirred in mixture ethyl acetate (120 mL), and n-heptane (700 mL) at 50-85° C. to get clear solution. Cooled to 25-30° C. Pure N-methoxycarbonyl-(L)-tert-leucine (0.2 g) was added, stirred for 1 hour, solid was filtered, washed with n-heptane and dried under reduced pressure to give 110 g of N-methoxycarbonyl-(L)-tert-leucine (D-isomer was below detection limit by HPLC).

### Example 2

### Preparation of Hydroxy compound (1-[4-(Pyridin-2-yl)-phenyl]-4(S)-hydroxy-5(S)-2,5-bis[(tert-butoxycarbonyl)amino]-6-phenyl-2-azahexane) (III)

N-1-(tert-butoxycarbonyl)-N-2-[4-(pyridine-2-yl-benzyl]-hydrazine (II) (100 g, 0.33 mole) and (2R)-[(1′S)-Boc-amino-2′-phenylethyl]oxirane (I) (102.8 g, 0.39 mole) were added in IPA (400 mL), and heated to reflux for 30 hours. Water (50 mL) was added slowly and stirred at 60-70° C. for 2 hours. Cooled to 15-20° C. and solid was filtered, washed with a mixture of IPA and water. Wet solid was crystallized from methanol-water, to give 160 g of hydroxy compound (III).

### Example 3a

### Preparation of Atazanavir Base (VI)

A) Hydroxy compound (III) (100.0 g, 0.18 mole) and concentrated HCl (68 mL) were added in dichloromethane (500 mL). Refluxed till completion of reaction. Cooled, and water was added. The aqueous layer containing HCl salt of diamino compound (IV) was separated.
B) To another flask N-methoxycarbonyl-(L)-tertiary leucine (V) (88.11 g, 0.47 mole), 1-(3-dimethylaminopropyl)-3-ethylcarbodiimide hydrochloride (EDC) (89.4 g, 0.47 mole), 1-Hydroxy-benzotriazole (HOBT) (75.5 g, 0.49 mole) and dichloromethane (1000 mL) were charged and stirred at 25-30° C. for 4-5 hours. The aqueous layer of diamino compound (IV) obtained above in part A, and N,N-diisopropylethyl amine (DIPEA) (182.8 mL, 138 g) were added and stirred for 3 hours. The reaction mass was then washed with water, sodium bicarbonate solution and brine. The dichloromethane layer was concentrated to 100-150 mL. Ethyl acetate (1000 mL) was added and about half of the mixture of solvent was distilled out. n-Heptane (400 mL) was added and stirred for 1 hour at 65° C. Cooled to 30° C., solid was filtered, washed with mixture of ethyl acetate and n-heptane and dried to afford 101 g of atazanavir base (crude).

### Example 3b

### Preparation of Atazanavir Base (VI) Using DCC as Coupling Agent

To another flask N-methoxycarbonyl-(L)-tertiary leucine (V) (88.11 g, 0.47 mole), N,N′-Dicyclohexylcarbodiimide (DCC) (96.2 g, 0.47 mole), 1-Hydroxy-benzotriazole (HOBT) (75.5 g, 0.49 mole) and dichloromethane (1000 mL) were charged and stirred at 25-30° C. for 4-5 hours. The aqueous layer of diamino compound (IV) obtained above in part A of example-3a, and N,N-diisopropylethyl amine (DIPEA) (182.8 mL, 138 g) were added and stirred for 3 hours. The reaction mass was then washed with water, sodium bicarbonate solution and brine. The dichloromethane layer was concentrated to obtain crude product which was further purified by column chromatography by using dichloromethane: methanol (98:2) as eluent to obtain a pure base (78 g)

### Example 4

### Purification of Atazanavir Base

Atazanavir base (100 Kg, 142 mole) was added in 700 L ethanol and stirred at 80-85° C. for 40-50 minutes. Water (700 L) was added in hot condition. Cooled to room temperature. Solid was filtered, washed with 1:1 mixture of ethanol-water and dried to afford 90 Kg of pure atazanavir base. (HPLC data: atazanavir—99.98%, RSSS isomer—0.01%, SSSR isomer—below detection limit, RSSR isomer—below detection limit).

### Example 5

### Preparation of Atazanavir Sulfate

To a solution of atazanavir base (60 Kg) in ethanol (390 L), concentrated sulfuric acid (5.16 L) was added at 25-30° C. and stirred for 40 minutes. To the solution n-heptane (498 L) and seed of atazanavir sulfate (180 g) were added. Stirred at 25-30° C. for 16 hours. The solid was filtered, washed with 1:1 mixture of ethanol: n-heptane and dried to give 58 Kg of atazanavir sulfate. (HPLC data: atazanavir sulfate—99.93%, RSSS isomer—0.01%, SSSR isomer—0.01%, RSSR isomer-below detection limit).

1. A process for preparation atazanavir sulfate of formula, 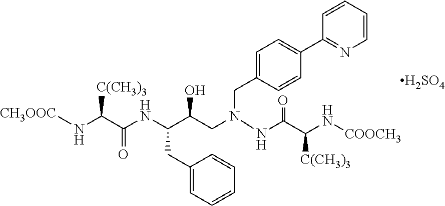
that is substantially free of its diastereomeric impurities comprising the steps:
a) reaction of diamino compound (IV) with N-methoxycarbonyl-L-tertiary leucine (V) having D-tertiary leucine isomer less than 0.1% to obtain atazanavir base (VI); 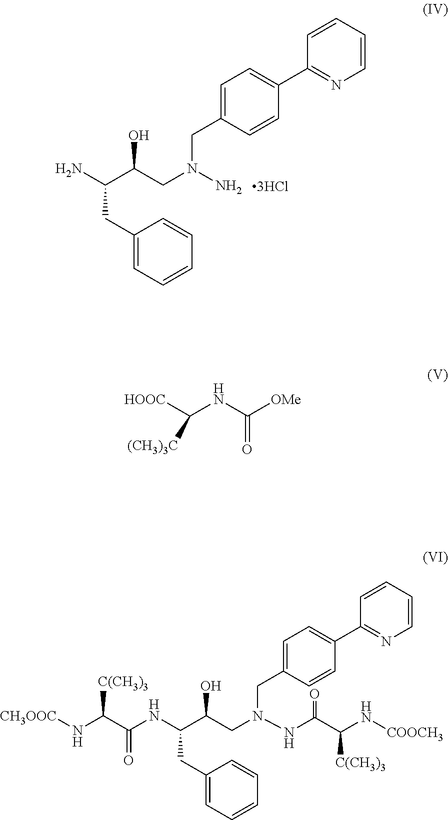
b) optionally purification of atazanavir base (VI); and
c) conversion of atazanavir base (VI) to atazanavir sulfate.

2. The process of claim 1, wherein diastereomeric impurities of atazanavir are less than 0.2%, measured as area percentage by HPLC.

3. The process of claim 1, wherein diastereomeric impurities of atazanavir are less than 0.1%, measured as area percentage by HPLC.

4. The process of claim 1, wherein diastereomeric impurities of atazanavir are less than 0.05%, measured as area percentage by HPLC.

5. The process of claim 1, wherein the step (a) is carried out in the presence of carbonyl activating agent, carbodiimide and organic tertiary-amine in a suitable solvent.

6. The process of claim 5, wherein the carbodiimide is water soluble carbodiimide such as 1-(3-dimethylaminopropyl)-3-ethylcarbodiimide hydrochloride.

7. The process of claim 5, wherein the carbodiimide is water insoluble carbodiimide selected from dicyclohexyl carbodiimide and diisopropyl carbodiimide, preferably dicyclohexyl carbodiimide.

8. The process of claim 5, wherein suitable solvent is water immiscible organic solvent or mixture of water immiscible organic solvent and water or mixture of water miscible organic solvent and water.

9. The process according to claim 8, wherein water immiscible solvent is selected from the group such as halogenated hydrocarbons like dichloromethane, chloroform, dichloroethane; esters like ethyl acetate, propyl acetate, butyl acetate; aromatic solvents like benzene, toluene, xylene, ethylbenzene, chlorobenzene; ethers like diethyl ether, diisopropyl ether and methyl tert-butylether; preferably dichloromethane.

10. Process of preparation according to claim 8, wherein water miscible solvent is selected from dimethyl acetamide, dimethyl formamide, tetrahydrofuran, dioxane, acetone, methyl isobutyl ketone, methylethyl ketone, acetonitrile and propionitrile; and mixtures thereof, preferably dimethyl formamide.

11. The process of claim 5, wherein the carbonyl activating agent is selected from 1-hydroxy-benzotriazole and 1-hydroxy-aza-benzotriazole, preferably 1-hydroxy-benzotriazole.

12. Process of claim 5, wherein the organic tertiary amine is selected from triethylamine, tert-butylamine, N,N-diisopropylethyl amine and the likes; the preferred organic tertiary amine is N,N-diisopropylethyl amine.

13. The process of claim 1, wherein step (b) is carried out by crystallization from ethanol-water mixture or the methods known in literature.

14. The process of claim 1, wherein step (c) is carried by treating atazanavir base with concentrated sulfuric acid in a suitable solvent selected from acetonitrile, acetone, ethanol and heptane or mixtures thereof; preferred solvent is ethanol-heptane mixture.

15. The process for preparation of N-methoxycarbonyl-L-tertiary-leucine (V) having D-tertiary leucine isomer less than 0.1% comprising the steps:
a) selection of L-tertiary-leucine containing D-isomer less than 0.5%;
b) conversion of L-tertiary-leucine to N-methoxycarbonyl-L-tertiary leucine (V);
c) purification of N-methoxycarbonyl-L-tertiary leucine (V).

16. The process of claim 15, wherein step (b) is carried by reaction of L-tertiary-leucine with reagent selected from methylchloroformate, dimethyldicarbonate and N-methoxycarbonylphthalimide, preferably methylchloroformate.

17. The process of claim 15, wherein step (b) is carried out in an aqueous inorganic base and suitable solvent.

18. The process of claim 17, wherein inorganic base is selected from bases such as sodium hydroxide, potassium hydroxide, sodium carbonate; preferably sodium hydroxide.

19. The process of claim 17, wherein the suitable solvent is selected from ethers like diethyl ether, diisopropyl ether, methyl tert-butylether, tetrahydrofuran and dioxane; preferably dioxane.

20. The process of claim 15, wherein step (c) carried out in solvents selected from hydrocarbons like n-heptane, halogenated hydrocarbons like dichloromethane, chloroform, dichloroethane; amides like dimethyl acetamide, dimethyl formamide; esters like ethyl acetate, propyl acetate, butyl acetate; ethers like diethyl ether, diisopropyl ether, methyl tert-butylether, tetrahydrofuran, dioxane; aromatic solvents like benzene, toluene, xylene, ethylbenzene, chlorobenzene; ketones like acetone, methyl isobutyl ketone, methylethyl ketone; nitriles like acetonitrile and propionitrile; and mixtures thereof; preferably, from ethyl acetate-heptane mixture.

21. Atazanavir sulfate obtained by the process of claim 1 having diastereomeric impurities less than 0.2%, measured as area percentage by HPLC.

22. Atazanavir sulfate obtained by the process of claim 1 having diastereomeric impurities less than 0.1%, measured as area percentage by HPLC.

23. Atazanavir sulfate obtained by the process of claim 1 having diastereomeric impurities less than 0.05%, measured as area percentage by HPLC.

24. Pharmaceutical composition comprising atazanavir sulfate according to claim 1, together with at least one pharmaceutically acceptable excipient.
